(19) United States
(12) Patent Application Publication
(10) Pub. No.: US 2012/0149919 A1
(43) Pub. Date: Jun. 14, 2012

(54) MALEIC ANHYDRIDE SYNTHESIS CATALYST PRECURSOR AND PROCESS FOR ITS PREPARATION

(75) Inventors: Stefan Altwasser, Wachenheim (DE); Cornelia Katharina Dobner, Ludwigshafen (DE); Hagen Wilmer, Ludwigshafen (DE); Frank Rosowski, Mannheim (DE)

(73) Assignee: BASF SE, Ludwigshafen (DE)

(21) Appl. No.: 13/391,958

(22) PCT Filed: Aug. 23, 2010
(86) PCT No.: PCT/EP2010/062207

§ 371 Date: Feb. 23, 2012

(30) Foreign Application Priority Data
Aug. 26, 2009 (EP) 09168703.8

(51) Int. Cl. B01J 27/198; C07D 307/60; C01B 25/37

(52) U.S. Cl. 549/256; 423/309; 502/209; 423/308

(57) ABSTRACT

A process for preparing a vanadium, phosphorus, and oxygen comprising catalyst precursor for preparing maleic anhydride by heterogeneously catalyzed gas-phase oxidation of a hydrocarbon having at least four carbon atoms, which comprises
(a) reacting vanadium pentoxide with 102% to 110% strength phosphoric acid in the presence of isobutanol and optionally of a primary or secondary, noncyclic or cyclic, unbranched or branched, saturated alcohol having 3 to 6 carbon atoms in a temperature range from 80 to 160° C.;
(b) isolating the precipitate formed;
(c) (i) drying the isolated precipitate down to a residual isobutanol content of less than 5% by weight;
   (ii) then passing a gas which in addition to one or more inert gases comprises 0.1% to 9% by volume of oxygen through the dried precipitate, directly or after isolation, in the temperature range from 130° C. to 200° C.

The present invention relates to a vanadium, phosphorus, and oxygen comprising catalyst precursor and also to a process for its preparation for preparing maleic anhydride by heterogeneously catalyzed gas-phase oxidation of a hydrocarbon having at least four carbon atoms.

The present invention further relates to a vanadium, phosphorus, and oxygen comprising catalyst and to a process for its preparation using the catalyst precursor of the invention.

The present invention additionally relates to a process for preparing maleic anhydride by heterogeneously catalyzed gas-phase oxidation of a hydrocarbon having at least four carbon atoms using the catalyst of the invention.

Maleic anhydride is an important intermediate in the synthesis of y-butyrolactone, tetrahydrofuran, and 1,4-butanediol, which in turn are used as solvents or are processed further, for example, to polymers, such as polytetrahydrofuran or polyvinylpyrrolidone.

The preparation of maleic anhydride by oxidizing hydrocarbons such as n-butane, n-butenes or benzene over suitable catalysts is well established. It is commonly carried out using vanadium-phosphorus-oxygen catalysts (known as VPO catalysts) (see Ullmann's Encyclopedia of Industrial Chemistry, 6^{th} edition, 2000 electronic release, Chapter “MALEIC AND FUMARIC ACIDS, Maleic Anhydride—Production”).

The vanadium-phosphorus-oxygen catalysts widely employed are generally prepared as follows:
(1) Synthesis of a vanadyl phosphate hemihydrate precursor (VOHPO₄×½ H₂O) from a pentavalent vanadium compound (e.g., V₂O₅), a pentavalent or trivalent phosphorus compound (e.g., orthophosphoric and/or pyrophosphoric acid, phosphoric esters or phosphorous acid), and a reductive alcohol (e.g., isobutanol), isolation of the precipitate, and drying, with shaping if desired (tableting for example); and
(2) preactivation to give vanadyl pyrophosphate ((VO)₂P₂O₇) by calcination.

Owing to the use of a reductive alcohol as reductant, several percent by weight of organic compounds generally remain included in the resultant precursor precipitate, and cannot be removed even by extensive washing. During the subsequent preparation of the catalyst, and especially during calcinationg, these organic compounds exert a negative effect on the catalytic properties of the catalyst. For instance, during the later calcination operation, there is a risk of these included organic compounds evaporating and/or undergoing thermal decomposition to form gaseous components which may lead to an increase in pressure inside the catalyst structure and hence to its destruction. This deleterious effect is particularly strongly pronounced when calcination is carried out under oxidative conditions, since the formation of the oxidized degradation products, such as carbon monoxide or carbon dioxide, produces a substantially greater volume of gas. Moreover, the oxidation of these organic compounds produces very large quantities of heat locally, which may lead to thermal damage to the catalyst.

Furthermore, the included organic compounds possess a significant influence on the local oxidation state of the vanadium. Thus in Chemie Ingenieur Technik 72(3), 2000, pages 249 to 251 B. Kubias et al. demonstrate the reductive effect of organic carbon in the anaerobic calcination (under nonoxidative conditions) of a vanadyl hydrogen phosphate hemihydrate precursor obtained from solution in isobutanol. In the example given, anaerobic calcination produced an average oxidation state of the vanadium of 3.1 whereas aerobic calcination (under oxidative conditions) produces an average oxidation state of the vanadium of approximately 4.

WO 99/67021 A1 (Pantochim) describes in example 1 the preparation of a catalyst precursor, where isobutyl alcohol, benzyl alcohol, vanadium pentoxide and 106% phosphoric acid are reacted at approximately 107° C. and the precipitate is filtered off and dried in open trays in a forced-draft oven at 150° C. for 10 hours. WO 99/67021 does not describe first drying the precipitate down to a defined isobutanol content and then passing a gas having a defined oxygen content through it at a defined temperature.

WO 95/29006 A1 (Pantochim) describes in example 1 the preparation of a catalyst precursor, where isobutyl alcohol, benzyl alcohol, vanadium pentoxide and 106% phosphoric acid are reacted at approximately 107° C. and the precipitate is filtered off and dried in open trays in a forced-draft oven at 150° C. for 10 hours. This powder is then shaped into cylindrical tablets which are subsequently calcined, in one instance in a 25 air/75 nitrogen mixture, with a temperature program from room temperature to 150° C. and on up to 420° C. WO 95/29006 does not describe first drying the precipitate down to a defined isobutanol content and then passing a gas having a defined oxygen content through it at a defined temperature.

It was an object of the present invention to find a process for preparing a vanadium, phosphorus, and oxygen comprising catalyst precursor for preparing maleic anhydride by heterogeneously catalyzed gas-phase oxidation of a hydrocarbon having at least four carbon atoms that no longer possesses the disadvantages set out above, is easy and economic to carry out industrially, and, following preactivation, which should likewise be easy to carry out industrially, gives a particulate catalyst, and which possesses high activity and high selectivity.

We have found that this object was achieved by a process for preparing a vanadium, phosphorus, and oxygen comprising catalyst precursor for preparing maleic anhydride by heterogeneously catalyzed gas-phase oxidation of a hydrocarbon having at least four carbon atoms, which comprises
(a) reacting vanadium pentoxide with 102% to 110% phosphoric acid in the presence of isobutanol and optionally of a primary or secondary, noncyclic or cyclic, unbranched or branched, saturated alcohol having 3 to 6 carbon atoms in a temperature range from 80 to 160° C.;
(b) isolating the precipitate formed;
(c) (i) drying the isolated precipitate down to a residual isobutanol content of less than 5% by weight;
   (ii) then passing a gas which in addition to one or more inert gases comprises 0.1% to 9% by volume of oxygen through the dry precipitate, directly or after isolation, in the temperature range from 130° C. to 200° C.

Steps (a) to (c) above are illustrated in more detail below:

Step (a)

The phosphoric acid for use in the process of the invention possesses an arithmetic H₃PO₄ content of 102% to 110% by weight. This is expressed for simplification as 102 to 106% phosphoric acid. The 102 to 110% phosphoric acid is a mixture comprising orthophosphoric acid (H₃PO₄), pyrophosphoric acid (H₄P₂O₇), and polyphosphoric acids of the general formula Hₙ₊₂PₙO₃ₙ₊₁ with n≧3. For the process of the invention it is preferred to use 102 to 108% phosphoric acid, with particular preference from 102 to 106%, and with very particular preference from 104 to 106%. The phosphoric acid for use is generally prepared by introducing phosphorus pentoxide into water or aqueous, for example 85 to 100% phosphoric acid.

The reductive component used in the process of the invention is isobutanol (2-methyl-1-propanol) and optionally a primary or secondary, noncyclic or cyclic, unbranched or branched, saturated alcohol having from 3 to 6 carbon atoms or a mixture of such alcohols. For the process of the invention it is preferred, as well as the isobutanol, to use a primary or secondary, unbranched or branched, C3 to C6 alkanol or to use cyclopentanol or cyclohexanol. Suitable alcohols include n-propanol (1-propanol), isopropanol (2-propanol), n-butanol (1-butanol), sec-butanol (2-butanol), 1-pentanol, 2-pentanol, 3-pentanol, 2-methyl-1-butanol, 3-methyl-1-butanol, 3-methyl-2-butanol, 2,2-dimethyl-1-propanol, 1-hexanol, 2-hexanol, 3-hexanol, 2-methyl-1-hexanol, 3-methyl-1-pentanol, 4-methyl-1-pentanol, 3-methyl-2-pentanol, 4-methyl-2-pentanol, 2,2-dimethyl-1-butanol, 2,3-dimethyl-1-butanol, 3,3-dimethyl-1-butanol, 3,3-dimethyl-2-butanol, cyclopentanol, cyclohexanol, and mixtures thereof. Used with particular preference, as well as isobutanol, are primary, unbranched or branched, C₃ to C₅ alkanols and also cyclohexanol. Very particular preference is given, as well as isobutanol, to using n-propanol (1-propanol), n-butanol (1-butanol), 1-pentanol, 2-methyl-1-butanol, 3-methyl-1-butanol, and cyclohexanol. With particular preference, the reductive component used in the process of the invention is 90% to 100% by weight of isobutanol; with very particular preference, only isobutanol (2-methyl-1-propanol) is used.

The measure of using a primary or secondary, noncyclic or cyclic, unbranched or branched, saturated alcohol having from 3 to 6 carbon atoms, as well as isobutanol, generally makes it easier to remove the alkanol or alkanol mixture and its decomposition products and hence to achieve a lower organic carbon content in the catalyst precursor after step c). In contrast, other reductants such as benzyl alcohol and its decomposition products from the reduction are much more difficult to remove, ultimately implying a disadvantageously high organic carbon content.

Furthermore, in the process of the invention, additional reductive components may be used as well. Examples include ethanol, formic acid, and oxalic acid.

The process of the invention is preferably conducted without adding additional reductive components.

The vanadium pentoxide is used generally in the form of a powder, with particular preference in a particle range from 50 to 500 micrometers. Where much larger particles are present, the solid is usually milled before being used and is sieved if desired. Examples of suitable apparatus are ball mills or planetary mills.

In the preparation of the catalyst precursor it is also possible to add what are known as promoter components. Suitable promoters are the elements of groups 1 to 15 of the Periodic System and also their compounds. Suitable promoters are described, for example, in WO 97/12674 and WO 95/26817 and also in U.S. Pat. No. 5,137,860, U.S. Pat. No. 5,296,436, U.S. Pat. No. 5,158,923, and U.S. Pat. No. 4,795,818. Preferred promoters are compounds of the elements cobalt, molybdenum, iron, zinc, hafnium, zirconium, lithium, titanium, chromium, manganese, nickel, copper, boron, silicon, antimony, tin, niobium and bismuth, with particular preference molybdenum, iron, zinc, antimony, bismuth, and lithium. The promoted catalysts may comprise one or more promoters. The promoter components are generally added during step (a), i.e., said reaction of the vanadium pentoxide with 102% to 110% phosphoric acid in the presence of a primary or secondary, noncyclic or cyclic, unbranched or branched, saturated alcohol having from 3 to 6 carbon atoms. The total promoter content of the finished catalyst is generally not more than about 5% by weight, calculated in each case as the oxide.

Where promoted catalyst precursors are prepared by the process of the invention, the promoter is generally added in the form of an organic or inorganic salt during the combining of the vanadium pentoxide, the 102 to 110% phosphoric acid, and the isobutanol and optionally the primary or secondary, noncyclic or cyclic, unbranched or branched, saturated alcohol having from 3 to 6 carbon atoms. Examples of suitable promoter compounds are the acetates, acetylacetonates, oxalates, oxides, and alkoxides of the aforementioned promoter metals, such as cobalt(II) acetate, cobalt(II) acetylacetonate, cobalt(II) chloride, molybdenum(VI) oxide, molybdenum(III) chloride, iron(III) acetylacetonate, iron(III) chloride, zinc(II) oxide, zinc(II) acetylacetonate, lithium chloride, lithium oxide, bismuth(III) chloride, bismuth(III) ethylhexanoate, nickel(II) ethylhexanoate, nickel(II) oxalate, zirconyl chloride, zirconium(IV) butoxide, silicon(IV) ethoxide, niobium(V) chloride, and niobium(V) oxide. For further details, refer to the aforementioned laid-open WO specifications and U.S. patents.

In the process of the invention, the combining of the vanadium pentoxide, the 102 to 110% phosphoric acid, and the isobutanol and optionally the primary or secondary, noncyclic or cyclic, unbranched or branched, saturated alcohol having from 3 to 6 carbon atoms can be accomplished in a variety of ways. For example, said components may be introduced initially or added, neat, in diluted form or, in the case of vanadium pentoxide, as a suspension. Dilution or suspension is generally carried out using the primary or secondary, noncyclic or cyclic, unbranched or branched, saturated alcohol having from 3 to 6 carbon atoms.

Said combining is generally carried out in the reaction apparatus suitable for the subsequent reaction—a stirred tank, for example—and with mixing. The components to be combined, in neat, diluted or suspended form, are generally conditioned to a temperature in the range from 0 to 160° C., although the components to be combined may of course possess a different temperature.

Examples of variants of the combining are described in WO 03/078058 A1 (BASF), pages 9 to 10.

The relative molar ratio of the 102 to 110% phosphoric acid to the vanadium pentoxide is generally set in accordance with the desired ratio in the catalyst precursor. In the reaction mixture for preparing the catalyst precursor, the molar phosphorus/vanadium ratio is preferably from 1.0 to 1.5 and with particular preference from 1.1 to 1.3.

The amount of the isobutanol and optionally the primary or secondary, noncyclic or cyclic, unbranched or branched, saturated alcohol having from 3 to 6 carbon atoms should advantageously be beyond the amount required stoichiometrically to reduce the vanadium from the oxidation state +5 to an oxidation state in the range from +3.5 to +4.5. The amount should also be such as to be able to form, with the vanadium pentoxide, a suspension which allows intensive mixing with the 102 to 110% phosphoric acid.

The molar ratio of the total amount of the isobutanol plus, where used, the primary or secondary, noncyclic or cyclic, unbranched or branched, saturated alcohol having 3 to 6 carbon atoms to the vanadium pentoxide is generally from 10 to 25 and preferably from 12 to 20.

When the vanadium pentoxide, 102-110% phosphoric acid, and primary or secondary, noncyclic or cyclic, unbranched or branched, saturated 3- to 6-carbon atom alcohol components have been combined, the reaction of these compounds is accomplished by heating the mixture at a temperature from 80 to 160° C. over a period of usually several hours. The temperature range to be selected is dependent on a variety of factors, one example being the boiling point of the added alcohol, and can be optimized by means of simple experiments. When using isobutanol alone, which is very particularly preferred, the mixture is heated preferably at a temperature from 90 to 120° C. and with particular preference from 100 to 110° C. The volatile compounds, such as water, the alcohol and its degradation products, such as aldehyde or carboxylic acid, for instance, generally evaporate from the reaction mixture and can either be taken off or else completely or partly condensed and recycled. Preference is given to complete or partial recycling by heating under reflux. Complete recycling is particularly preferred. The reaction at elevated temperature generally lasts several hours and is dependent on a large number of factors, such as the nature of the added components or the temperature, for example. Moreover, within a certain range, the temperature and the selected heating period may also be used to set and influence the properties of the catalyst precursor. For a given system, the parameters of temperature and time can be optimized simply by means of a few experiments. The time taken for said reaction is customarily from 1 to 25 hours.

Step (b)

After the end of the reaction, the precipitate formed is isolated, where appropriate following a cooling phase and also a storage or aging phase of the cooled reaction mixture. In the isolation procedure, the precipitate is separated off from the liquid phase. Examples of suitable methods are filtration, decanting, and centrifugation. The precipitate is preferably isolated by filtering or centrifuging. Isolation of the precipitate takes place generally likewise within a temperature range from 0 to 160° C. with temperatures in the range from 50 to 150° C., in particular from 80 to 150° C., being preferred.

The isolated precipitate can be processed further with or without washing. Washing the isolated precipitate has the advantage that adhering residues of the alkanol and its degradation products can be reduced further in amount. Examples that may be given of suitable solvents for the washing operation include alcohols (e.g., methanol, ethanol, 1-propanol, 2-propanol, and the primary or secondary, noncyclic or cyclic, unbranched or branched, saturated alcohol having from 3 to 6 carbon atoms that was selected for the preceding reaction), aliphatic and/or aromatic hydrocarbons (e.g., pentane, hexane, petroleum spirits, benzene, toluene, xylenes), ketones (e.g., 2-propanone (acetone), 2-butanone, 3-pentanone, ethers (e.g., 1,2-dimethoxyethane, tetrahydrofuran, 1,4-dioxane) or mixtures thereof. Where the isolated precipitate is washed, it is preferred to use the primary or secondary, noncyclic or cyclic, unbranched or branched, saturated alcohol having from 3 to 6 carbon atoms that was selected for the preceding reaction.

Step (c)(i)

The isolated precipitate is then dried. Drying may be carried out under various conditions. In general it is carried out at atmospheric pressure (0.1 MPa abs) or a lower pressure.

The drying temperature is generally 130 to 200° C., and, if drying under reduced pressure, it is possible in many cases to use lower temperatures than when drying under atmospheric pressure.

Preferably the drying is carried out at a low pressure in the range from 1 to 30 kPa in the temperature range from 130 to 200° C.

The gas atmosphere that may be present over the material at the drying stage may comprise, in a very small amount, oxygen (generally less than 0.5% by volume of oxygen), water vapor and/or inert gases, such as nitrogen, carbon dioxide or noble gases, for instance. Drying is carried out preferably at a pressure of 1 to 30 kPa abs and a temperature of 50 to 200° C. under a low-oxygen (generally less than 0.5% by volume of oxygen) or very preferably, a virtually oxygen-free residual gas atmosphere—in nitrogen, for example.

Drying can be carried out for example in the filtration device itself or in a separate apparatus, as for example in a drying oven or a continuous belt drier.

The isolated and dried precipitate has an isobutanol content of less than 5% by weight, preferably of less than 2% by weight. The isobutanol content is determined as described in the examples.

Step (c)(ii)

The precipitate obtained in step (c)(i) can be subjected to further treatment preferably in the vessel in which it was dried as per (c)(i), as described below. Alternatively it may be isolated and subjected to further treatment in a different vessel, as described below. In both variants, the further treatment may take place directly or shortly after drying, or else the precipitate obtained in step (c)(i) may be subjected to further treatment following packing and, if desired, transport and/or storage, as described below.

The precipitate obtained in step (c)(i) is preferably subjected to further treatment in the same vessel directly after drying, as described below.

A gas which comprises, in addition to one or more inert gases, 0.1% to 9%, preferably 2% to 7%, by volume of oxygen is passed, preferably homogeneously, through the precipitate obtained in step (c)(i), in the temperature range from 130 to 200° C.

Inert gases here are those which under the stated conditions do not enter into any chemical reaction. Examples of such inert gases are the noble gases, argon, for example, and with particular preference, nitrogen.

Homogeneous passage herein denotes the fact that the height of the precipitate over its cross section is as far as possible homogeneous, and hence that the pressure drop over the precipitate is likewise as homogeneous as possible.

The volume flow rate of the gas mixture described is generally in the range from 10 to 500 Nm³/h.

The treatment is generally carried out until the isobutanol content described below has been reached.

The resulting precipitate generally has an isobutanol content in the range from 0.0% to 0.1% by weight. The isobutanol content is determined as described in the examples.

In step (c)(ii), an organic carbon content in the range from 2 to 3% by weight is generally set in the precipitate, the heat-treated product, following the addition of 3.0% by weight of graphite as internal standard and using CuK-alpha radiation (π=1.54×10⁻¹⁰ m), giving a powder X-ray diffraction diagram which in the 2θ region features a ratio of the height of the peak of any pyrophosphate phase present at 28.5° to the height of the peak due to graphite at 26.6° of ≦0.1. The method is described in the examples.

Organic carbon here is any carbon which cannot be removed from the powderous sample by adding 10% by weight aqueous hydrochloric acid and then heating the mixture while passing a stream of nitrogen through it. The organic carbon content is calculated from the difference between the total carbon content and the inorganic carbon content.

In order to determine the total carbon content, a powderous sample of precise weight is introduced in the presence of a stream of pure oxygen into a quartz tube heated at about 1000° C., the sample is calcined, and the carbon dioxide present in the combustion gas is quantified. By calculating back from the amount of carbon dioxide detected and the initial mass of the sample, it is then possible to determine the total carbon content. A precise description of the method is given in the examples under “Determination of the total carbon content”.

In order to determine the inorganic carbon content, a powderous sample of precise weight is admixed with 10% by weight aqueous hydrochloric acid, the carbon dioxide evolved is expelled by slow heating while passing a stream of nitrogen through it, and the removed carbon dioxide is quantified. By calculating back from the amount of carbon dioxide detected and the initial sample mass, it is then possible to calculate the inorganic carbon content. A precise description of the method is given in the examples under “Determination of the inorganic carbon content”.

In the process of the invention, the treatment in step (c)(ii) generally sets an organic carbon content in the range from 1 to 5% by weight, preferably 2 to 3% by weight.

In the process of the invention, the treatment in step (c)(ii) generally sets an inorganic carbon content, as determined by the method as described in the examples, of commonly less than 0.01% by weight.

Through the measure of setting a low organic carbon content in the range from 1 to 5% by weight, preferably in the range from 2 to 3% by weight, damage to the catalyst is minimized, or prevented, generally, in the course of subsequent calcination of the particulate catalyst precursor, and the setting of a very uniform vanadium oxidation state throughout the entire catalyst body is made possible.

The X-ray diffraction (XRD) diagram shows the intensity of the diffracted X-ray (in counts per second, cps) as a function of twice the diffraction angle, 2Θ. The powder XRD diagram is recorded using the powderous precipitate intimately mixed with 3% by weight of graphite. Recording of the powder XRD diagram takes place with a powder diffractometer with adjustable aperture and collimator, measurements being made in reflection mode. Each peak height is a product of the difference between the maximum intensity of the respective signal and the measured background. A precise description of the method is given in the examples under “X-ray-diffractometric analysis of the heat-treated precipitate”.

Typically the catalyst precursor obtained in (c)(ii) is additionally shaped and activated generally by calcination.

The shaping of the catalyst takes place as described below under (d).

Step (d)

In step (d), the product obtained from step (c) can be shaped into particles having an average diameter of at least 2 mm, preferably having a diameter in the range from 10 mm to 2 mm. The average diameter of a particle means the average value of the smallest and the largest dimensions between two plane-parallel plates.

By particles are meant not only irregularly shaped particles but also geometrically shaped particles, referred to as moldings.

The product obtained from step (c) is preferably shaped to form moldings.

Examples of suitable moldings include tablets, cylinders, hollow cylinders, beads, strands, wagon wheels, and extrudates.

High suitability is possessed, for example, by the following moldings (also referred to below as “multihole moldings”) having a substantially cylindrical structure with more than one internal coaxial bore, it being possible for the internal bores to have any kind of cross-sectional geometry—round, oval, or angular, for example—and for these bores to be present uniformly (for example only round) or in any kind of mixture (for example round and oval) in the molding.

Other forms of the moldings, such as trilobes and tristars (see WO 93/01155) or moldings having at least one notch on the outside (see U.S. Pat. No. 5,168,090) for example are likewise possible.

Where the shaping of the product obtained in (c) takes place by tableting, as is customary, for example, in the production of tablets, cylinders, and hollow cylinders, it is normal to add a tableting aid to the powder and to intimately mix the two components. Tableting aids are generally catalytically inert and enhance the tableting properties of the powder, by increasing the sliding properties and free-flow properties, for example. One suitable, preferred tableting aid is graphite. The added tableting aids generally remain within the activated catalyst. The amount of tableting aid in the finished catalyst is typically from about 2 to 6% by weight.

Particular preference is given to moldings having a substantially hollow cylindrical structure, and to the above-described multihole moldings.

A substantially hollow cylindrical structure is a structure which substantially comprises a cylinder having a continuous aperture between the two end faces. The cylinder is characterized by two substantially parallel end faces and a lateral surface, the cross section of the cylinder, i.e., parallel to the end faces, being substantially of circular structure. The cross section of the continuous aperture, i.e., parallel to the end faces of the cylinder, is likewise substantially of circular structure. The continuous aperture is preferably located centrally with respect to the end faces, although this is not to exclude other spatial arrangements.

A multihole molding is a structure which substantially comprises a cylinder having more than one coaxial aperture (“internal bore”) between the two end faces. The cylinder is characterized by two substantially parallel end faces and a lateral surface, the cross section of the cylinder, i.e. parallel to the end faces, being substantially of circular structure. The cross-sectional geometry of the continuous apertures (“internal bores”), i.e. parallel to the end faces of the cylinder, is arbitrary, being, for example, substantially of circular, oval or angular geometry. The more than one, preferably 2 to 20, more preferably 3 to 6, continuous apertures are preferably located arranged about the axis of the cylinder, preferably symmetrically.

The phrase “substantially” indicates that deviations from the ideal geometry, such as slight deformations in the circular structure, end faces which are not in plane-parallel alignment, flaked-off angles and edges, surface roughness or notches in the lateral surface, in the end faces, or in the inner surface of the continuous bore, for example, are included in the catalyst of the invention. Within the bounds of the accuracy of the tableting art, circular end faces, a circular cross section of the continuous bore, end faces in parallel alignment, and macroscopically smooth surfaces are preferred.

The substantially hollow cylindrical structure and the multihole molding can be described by an external diameter d₁, a height h as the distance between the two end faces, and an inner-hole (continuous-aperture) diameter d₂ and/or the inner holes. The external diameter d₁ is preferably from 3 to 10 mm, with particular preference from 4 to 8 mm, with very particular preference from 4.5 to 6 mm. The height h is preferably from 1 to 10 mm, with particular preference from 2 to 6 mm, with very particular preference from 2 to 5 mm.

For highly suitable moldings having a substantially hollow-cylindrical structure, the following is true: the continuous-aperture diameter d₂ is preferably from 1 to 8 mm, with particular preference from 2 to 6 mm, with very particular preference from 2 to 3 mm. Particular preference is given to a hollow cylindrical structure which features (a) a ratio of the height h to the continuous-aperture diameter d₂ of not more than 1.5 and (b) a ratio of the geometric surface area A_{geo} to the geometric volume V_{geo} of at least 2 mm⁻¹, as is described, for instance, in WO 01/68245.

For highly suitable multihole moldings the following is true: the diameter of the more than one continuous apertures d₂ is preferably 0.5 to 3.0 mm, more preferably 1.0 to 2.5 mm, very preferably 1.5 to 2.5 mm, it not being necessary for every aperture to have the same diameter. The stated apertures preferably have substantially circular cross-sectional geometry.

The substantial to complete avoidance of the formation of the pyrophosphate phase in the precipitate in step (c) and the shaping in step (d) prior to actual calcination, i.e., the conversion of the vanadyl hydrogen phosphate hemihydrate phase (VOHPO₄×½ H₂O) into the catalytically active pyrophosphate phase ((VO)₂P₂O₇), with elimination of water, surprisingly gives a catalyst structure which is more advantageous as far as the catalytic properties are concerned than in the case of shaping carried out after said phase conversion.

The invention further provides a catalyst precursor for preparing maleic anhydride by heterogeneously catalyzed gas-phase oxidation of a hydrocarbon having at least four carbon atoms, said precursor being obtainable by the process of the invention described above.

The process of the invention makes it possible to prepare a vanadium, phosphorus, and oxygen comprising catalyst precursor for preparing maleic anhydride by heterogeneously catalyzed gas-phase oxidation of a hydrocarbon having at least four carbon atoms, the catalyst precursor being easy to prepare.

The invention further provides a process for preparing a vanadium, phosphorus, and oxygen comprising catalyst for preparing maleic anhydride by heterogeneously catalyzed gas-phase oxidation of a hydrocarbon having at least four carbon atoms by treating a vanadium, phosphorus, and oxygen comprising catalyst precursor in at least one atmosphere comprising oxygen (O₂), hydrogen oxide (H₂O), and/or inert gas in a temperature range from 250 to 600° C., which comprises using as catalyst precursor a catalyst precursor of the invention as described above.

Examples of suitable inert gases that may be mentioned include nitrogen, carbon dioxide, and noble gases.

Calcination (also called “preactivation”) can be carried out batchwise, in a shaft furnace, tray furnace, muffle furnace or oven, for example, or continuously, in a rotary tube, belt kiln or rotary sphere kiln, for example. It may comprise successive, different sections as far as temperature is concerned, such as heating, temperature hold, or cooling, and successive, different sections as far as the atmospheres are concerned, such as oxygenous, steam-containing or oxygen-free gas atmospheres. Suitable preactivation processes are described, for example, in patents U.S. Pat. No. 5,137,860 and U.S. Pat. No. 4,933,312 and in the laid open specification WO 95/29006, which are expressly hereby incorporated but without limitation. Particular preference is given to continuous calcination in a belt kiln having at least two—for example, from two to ten—calcination zones, where appropriate with different gas atmospheres and different temperatures. By means of an appropriate combination of temperatures, treatment times, and gas atmospheres, adapted to the respective catalyst system, it is possible to influence and hence to tailor the mechanical and catalytic property of the catalyst.

Preferred in the process of the invention is a calcination wherein the catalyst precursor
(i) is heated to a temperature from 200 to 350° C. in at least one calcination zone in an oxidizing atmosphere having an oxygen content of from 2 to 21% by volume and is left under these conditions until the vanadium has the desired average oxidation state; and
(ii) is heated to a temperature from 300 to 500° C. in at least one further calcination zone in a nonoxidizing atmosphere having an oxygen content of ≦0.5% by volume and a hydrogen oxide content of from 20 to 75% by volume, and is left under these conditions for 0.5 hour.

In step (i), the catalyst precursor is left in an oxidizing atmosphere having a molecular oxygen content of generally from 2 to 21% by volume and preferably from 5 to 21% by volume at a temperature from 200 to 350° C. and preferably from 250 to 350° C. for a period effective to set the desired average oxidation state of the vanadium. Step (i) generally employs mixtures of oxygen, inert gases (e.g., nitrogen or argon), hydrogen oxide (steam) and/or air, and also air itself. From the standpoint of the catalyst precursor which is passed through the calcination zone or zones, the temperature during calcination step (i) may be kept constant or may on average rise or fall. Since step (i) is generally preceded by a heating phase, the temperature will generally rise first of all before then leveling off at the desired end value. In general terms, therefore, the calcination zone of step (i) is preceded by at least one further calcination zone in which the catalyst precursor is heated.

The time for which the heat treatment in step (i) is maintained is preferably selected, in the process of the invention, so as to set an average vanadium oxidation state at a value of from +3.9 to +4.4, preferably from +4.0 to +4.3. The average oxidation state of the vanadium is determined by potentiometric titration in accordance with the method described in the examples.

Since for reasons associated with the apparatus and with time it is extremely difficult to determine the average oxidation state of the vanadium during the calcination operation, the time period required is advantageously determined in preliminary experiments. This purpose is typically served by a series of measurements wherein heat treatment is carried out under defined conditions, with the samples being removed from the system after different times, cooled, and analyzed for the average oxidation state of the vanadium.

The time period required in the case of step (i) is generally dependent on the nature of the catalyst precursor, on the set temperature, and on the selected gas atmosphere, particularly on the oxygen content. Generally speaking, the time period for step (i) extends to a duration of more than 0.5 hour and preferably of more than 1 hour. A period of up to 4 hours, preferably of up to 2 hours, is generally sufficient to set the desired average oxidation state. Under appropriately adjusted conditions (e.g., lower range of the temperature span and/or low molecular oxygen content), however, a period of more than 6 hours may also be necessary.

In step (ii), the resultant catalyst intermediate is left in a nonoxidizing atmosphere having a molecular oxygen content of <0.5% by volume and a hydrogen oxide (steam) content of from 20 to 75% by volume, preferably from 30 to 60% by volume, at a temperature from 300 to 500° C. and preferably from 350 to 450° C. for a period of ≧0.5 hour, preferably from 2 to 10 hours, and with particular preference from 2 to 4 hours. In addition to the hydrogen oxide stated, the nonoxidizing atmosphere generally comprises predominantly nitrogen and/or noble gases, such as argon, for example, but without this constituting any restriction. Gases such as carbon dioxide, for example, are also suitable in principle. The nonoxidizing atmosphere preferably comprises ≧40% by volume of nitrogen. From the standpoint of the catalyst precursor which is passed through the calcination zone or zones, the temperature during calcination step (ii) may be held constant or may on average rise or fall. Where step (ii) is conducted at a higher or lower temperature than step (i), there is typically a heating or cooling phase between steps (i) and (ii) which is implemented where appropriate in a further calcination zone. In order to allow improved separation from the oxygenous atmosphere of step (i), said further calcination zone between (i) and (ii) may be flushed with inert gas, such as nitrogen, for example. Step (ii) is preferably conducted at a temperature which is higher by from 50 to 150° C. than that of step (i).

Generally speaking, calcination comprises a further step (iii), to be carried out later than step (ii), wherein the calcined catalyst precursor is cooled under an inert gas atmosphere to a temperature of ≦300° C., preferably ≦200° C., and with particular preference ≦150° C.

Before, between and/or after steps (i) and (ii), or (i), (ii), and (iii), further steps are possible when calcination in accordance with the process of the invention. Without limitation, further steps that may be mentioned include, for example, changes in temperature (heating, cooling), changes in the gas atmosphere (changeover of gas atmosphere), further holding times, transfers of the catalyst intermediate to different apparatus, or interruptions to the overall calcination operation.

Since the catalyst precursor generally has a temperature of <100° C. before calcination begins, it must normally be heated prior to step (i). Heating can be carried out using different gas atmospheres. Heating is preferably conducted in an oxidizing atmosphere, as defined under step (i), or in an inert gas atmosphere, as defined under step (iii). A change in gas atmosphere during the heating phase is also possible. Particular preference is given to heating in the oxidizing atmosphere which is also employed in step (i).

The invention further provides a catalyst for preparing maleic anhydride by heterogeneously catalyzed gas-phase oxidation of a hydrocarbon having at least four carbon atoms, said catalyst being obtainable by the process of the invention described above.

The catalyst prepared preferably by the process of the invention features a phosphorus/vanadium atomic ratio of from 0.9 to 1.5, with particular preference from 0.9 to 1.2, and with very particular preference from 1.0 to 1.1, an average vanadium oxidation state of from +3.9 to +4.4 and with particular preference from 4.0 to 4.3, a BET surface area of from 10 to 50 m²/g and with particular preference from 20 to 40 m²/g, a pore volume of from 0.1 to 0.5 ml/g and with particular preference from 0.2 to 0.4 ml/g, and a bulk density of from 0.5 to 1.5 kg/l and with particular preference from 0.5 to 1.0 kg/l.

The catalyst obtainable by calcination the catalyst precursor of the invention is distinguished by a substantially homogeneous oxidation state of the vanadium within the individual catalyst particles and between the different catalyst particles. In the heterogeneously catalyzed gas-phase oxidation of a hydrocarbon having at least four carbon atoms to maleic anhydride, the catalyst of the invention allows a high hydrocarbon space velocity in combination with high conversion, high activity, high selectivity, and a high space-time yield.

The invention additionally provides a process for preparing maleic anhydride by heterogeneously catalyzed gas-phase oxidation of a hydrocarbon having at least four carbon atoms with oxygenous gases, which comprises using a catalyst of the invention as described above.

In the process of the invention for preparing maleic anhydride, the reactors used are generally shell and tube reactors. Suitable hydrocarbons are generally aliphatic and aromatic, saturated and unsaturated hydrocarbons having at least four carbon atoms, such as 1,3-butadiene, 1-butene, 2-cis-butene, 2-trans-butene, n-butane, C₄ mixture, 1,3-pentadiene, 1,4-pentadiene, 1-pentene, 2-cis-pentene, 2-trans-pentene, n-pentane, cyclopentadiene, dicyclopentadiene, cyclopentene, cyclopentane, C5 mixture, hexenes, hexanes, cyclohexane, and benzene, for example. Used with preference are 1-butene, 2-cis-butene, 2-trans-butene, n-butane, benzene or mixtures thereof. Particular preference is given to using n-butane and liquids and gases containing n-butane. The n-butane used may come, for example, from natural gas, from steam crackers or from FCC crackers.

The addition of the hydrocarbon generally takes place under flow control, i.e., with continuous introduction of a defined amount per unit time. The hydrocarbon can be metered in liquid or gaseous form. It is preferably metered in liquid form with subsequent vaporization before entering the shell and tube reactor.

Oxidants used are oxygenous gases, such as air, synthetic air, an oxygen-enriched gas, or else what is termed “pure” oxygen, i.e., oxygen originating, for example, from the fractionation of air. The oxygenous gas as well is added under flow control.

The gas to be passed through the shell and tube reactor generally contains a hydrocarbon concentration of from 0.5 to 15% by volume and an oxygen concentration of from 8 to 25% by volume. The remaining fraction is composed of other gases such as nitrogen, noble gases, carbon monoxide, carbon dioxide, steam, oxygenated hydrocarbons (e.g., methanol, formaldehyde, formic acid, ethanol, acetyaldehyde, acetic acid, propanol, propionaldehyde, propionic acid, acrolein, and crotonaldehyde) and mixtures thereof. As a proportion of the total amount of hydrocarbon, the n-butane fraction is preferably 90% and with particular preference 95%.

In order to ensure a long catalyst life and a further increase in conversion, selectivity, yield, space velocity over the catalyst and space-time yield, in the process of the invention it is preferred to supply a volatile phosphorus compound to the gas. The concentration of this compound at the beginning, i.e., at the reactor entry, is at least 0.2 ppm by volume, i.e., 0.2×10⁻⁶ parts by volume of the volatile phosphorus compounds relative to the total volume of the gas at the reactor entry. Preference is given to an amount of from 0.2 to 20 ppm by volume, with particular preference from 0.5 to 10 ppm by volume. Volatile phosphorus compounds are all phosphorus compounds which are present in gaseous form at the desired concentration under the conditions in which they are used. Examples of suitable volatile phosphorus compounds include phosphines and phosphoric esters. Particular preference is given to the C₁ to C₄ alkyl phosphates, with very particular preference trimethyl phosphate, triethyl phosphate, and tripropyl phosphate, and especially triethyl phosphate.

The process of the invention is generally conducted at a temperature from 350 to 480° C. The temperature referred to here is the temperature of the catalyst bed in the shell and tube reactor which said bed would be at were the process to be performed in the absence of a chemical reaction. If this temperature is not exactly the same at every point, then the term refers to the numerical average of the temperatures along the reaction zone. This means in particular that the true temperature prevailing at the catalyst may even lie outside of the stated range, owing to the exothermic nature of the oxidation reaction. The process of the invention is preferably conducted at a temperature from 380 to 460° C., with particular preference from 380 to 430° C.

The process of the invention can be performed at a pressure below atmospheric pressure (e.g., up to 0.05 MPa abs) or else above atmospheric pressure (e.g., up to 10 MPa abs). The pressure referred to here is the pressure within the shell and tube reactor unit. A pressure of from 0.1 to 1.0 MPa abs is preferred, with particular preference from 0.1 to 0.5 MPa abs.

The process of the invention can be carried out in two preferred modes, the single pass mode and the recycling mode. In the single pass mode, maleic anhydride and any oxygenated hydrocarbon byproducts are subtracted from the reactor discharge and the remaining gas mixture is removed and employed where appropriate for generating heat. In the case of the recycling mode, maleic anhydride and any oxygenated hydrocarbon byproducts are again subtracted from the reactor discharge and some or all of the remaining gas mixture, which contains unreacted hydrocarbon, is recycled to the reactor. Another variant of the recycling mode involves removing the unreacted hydrocarbon and recycling it to the reactor.

In one particularly preferred embodiment for the preparation of maleic anhydride, n-butane is the starting hydrocarbon used and the heterogeneously catalyzed gas-phase oxidation is conducted in a single pass over the catalyst of the invention.

The process of the invention using the catalysts of the invention allows a high hydrocarbon space velocity of the catalyst in combination with a high conversion rate owing to a high activity. The process of the invention also permits high selectivity and a high yield of maleic anhydride.

### DEFINITIONS

The variables used in this specification are, unless mentioned otherwise, defined as follows:
Conversion U=n(HC, reactor entry)−n(HC, reactor exit/n(HC, reactor entry)
Selectivity S=n(MAn, reactor exit)/n(HC, reactor entry)−n(HC, reactor exit)
Yield A=U×S
   and the abbreviations, etc. have the following definitions:
“x” =multiplied by
m(MAn)=mass of maleic anhydride (MAn) produced [g]
V(catalyst)=bed volume of catalyst, totaled over all reaction zones [I]
T=time unit [h]

V(HC)=volume of hydrocarbon in the gas phase, standardized to 0° C. and 0.1013 MPa [I(stp)] (Arithmetic variable. Where a hydrocarbon is in the liquid phase under these conditions, the hypothetical gas volume is calculated using the ideal gas law.)
U=conversion of hydrocarbons per reactor pass
S=selectivity for maleic anhydride per reactor pass
A=yield of maleic anhydride per reactor pass
n(HC)=amount-of-substance flow of hydrocarbons at the reactor entry [mol/h]
n(HC, reactor entry)=amount-of-substance flow of hydrocarbons at the reactor entry [mol/h]
n(HC, reactor exit)=amount-of-substance flow of hydrocarbons at the reactor exit [mol/h]
n(HC, plant entry)=amount-of-substance flow of hydrocarbons at the entry to the plant [mol/h]
n(HC, plant exit)=amount-of-substance flow of hydrocarbons at the exit of the plant [mol/h]
n(MAn, reactor entry)=amount-of-substance flow of maleic anhydride at the reactor entry [mol/h]
n(MAn, reactor exit)=amount-of-substance flow of maleic anhydride at the reactor exit [mol/h]
n(MAn, plant exit)=amount-of-substance flow of maleic anhydride at the plant exit [mol/h].

### EXAMPLES

Determination of the residual isobutanol content of the dried catalyst precursor

In order to determine the residual isobutanol content, about 4 g of the dried powderous catalyst precursor and about 10 g of N,N-dimethylformamide were weighed out precisely into a heatable stirred apparatus with reflux condenser. The mixture was then heated to boiling temperature with stirring and left under these conditions for 30 minutes. After cooling, the suspension was filtered and the isobutanol content of the filtrate was quantified by gas chromatography. The residual isobutanol content was then calculated from the concentration of isobutanol found in the N,N-dimethylformamide and from the initial weights of N,N-dimethylformamide and catalyst precursor.

Determination of the Total Carbon Content

In order to determine the total carbon content, approximately 50 to 200 mg of the powderous sample, weighed out precisely, were introduced in the presence of a stream of pure oxygen into a quartz tube heated to about 1000° C., and calcined. The combustion gas obtained was passed through an IR cell and the carbon dioxide content was quantified. Working back from the amount of carbon dioxide detected, it was possible to calculate the total carbon content of the sample.

Determination of the Inorganic Carbon Content

In order to determine the inorganic carbon content, about 50 to 200 mg of the powderous sample, weighed out precisely, were admixed with 10% by weight aqueous hydrochloric acid. The carbon dioxide evolved was removed with slow heating, during which a stream of nitrogen was passed through the mixture, and was purified by passing it through a cascade comprising a cold trap cooled with isopropanol/dry ice, two absorption vessels containing potassium permanganate solution, one absorption vessel containing concentrated sulfuric acid, and a manganese dioxide tube. The purified stream of gas was passed into a coulometer cell which was filled with a solution of 0.1% by weight thymolphthalein in dimethyl sulfoxide, and the change in color was monitored photometrically. From the change in transmission it is possible to deduce the amount of carbon dioxide introduced and hence the inorganic carbon content of the sample.

Determination of the Organic Carbon Content

The organic carbon content is calculated from the difference between the total carbon content and the inorganic carbon content.

X-Ray-Diffractometric Analysis of the Powder

For XRD analysis, the powder intimately mixed with 3% by weight of graphite was subjected to measurement in a Siemens D5000 theta/theta X-ray powder diffractometer. The measurement parameters were as follows:

| Circle diameter | 435 | mm |
|---|---|---|
| X-rays | CuK-alpha | |
| | (λ = 1.54 × 10−¹⁰ m) | |
| Tube voltage | 40 | kV |
| Tube current | 30 | mA |
| Aperture | variable V20 | |
| Collimator | variable V20 | |
| Secondary monochromator | graphite | |
| Monochromator aperture | 0.1 | mm |
| Detector aperture of | 0.6 | mm |
| scintillation counter | | |
| Step width | 0.02° | 2θ |
| Step mode | continuous | |
| Measurement time | 2.4 | s/step |
| Measurement rate | 0.5° | 2θ/min |

Each peak height is given by the difference between the maximum intensity of the respective signal and the measured background.

Determination of the Lateral Mechanical Strength of the Hollow Cylinders

In order to determine the lateral compressive strength, the hollow cylinders were placed in each case by the rounded side face onto the planar metal platform of a corresponding measuring device, in successive measurements. The two plane-parallel end faces were therefore in the vertical direction. A planar metal die was then lowered onto the hollow cylinder at a rate of advance of 1.6 mm/min and the progress of the force acting on the hollow cylinder was recorded until the cylinder fractured. The lateral compressive strength of each individual hollow cylinder corresponds to the maximum force.

The lateral compressive strength was determined by averaging the result of 30 individual measurements.

Determination of the Average Oxidation State of the Vanadium

The average oxidation state of the vanadium was determined by potentiometric titration.

For the determination, from 200 to 300 mg of each sample were introduced under argon atmosphere into a mixture of 15 mL of 50% sulfuric acid and 5 mL of 85% phosphoric acid, and dissolved with heating. The solution was subsequently transferred to a titration vessel fitted with two Pt electrodes. Each titration was carried out at 80° C. A titration with 0.1 molar potassium permanganate solution was carried out first. Where there were two steps resulting in the potentiometric curve, the vanadium was present in an average oxidation state of from +3 to less than +4. Where only one step was obtained, the vanadium was in an oxidation state of from +4 to less than +5.

In the first-mentioned case (two steps/+3≦Vₒₓ<+4) the solution contained no V⁵⁺; in other words, all of the vanadium was detected titrimetrically. The amount of V³⁺ and V⁴⁺ was calculated from the consumption of 0.1 molar potassium permanganate solution and the position of the two steps. The weighted average then gave the average oxidation state.

In the second-mentioned case (one step/+4≦Vₒₓ<+5), the amount of V⁴⁺ was calculated from the consumption of 0.1 molar potassium permanganate solution. By then reducing the total V⁵⁺ in the resulting solution with a 0.1 molar ammonium iron(II) sulfate solution and carrying out oxidation again with 0.1 molar potassium permanganate solution, the total amount of vanadium was calculated. The difference between the total amount of vanadium and the amount of V⁴⁺ gave the amount of V⁵⁺ originally present. The weighted average then gave the average oxidation state.

A) Preparation of the Catalyst Precursor Powder

### Example 1a

### Inventive

An 8 m³-steel/enamel stirred tank, rendered inert with nitrogen, externally heatable by way of pressurized water, and containing flow breakers was charged with 4602 kg of isobutanol. After the three-stage impeller stirrer had been started up, the isobutanol was heated to 90° C. under reflux. On reaching this temperature, the addition of 690 kg of vanadium pentoxide was commenced by way of the conveying screw. When, after about 20 minutes, about ⅔ of the desired amount of vanadium pentoxide had been added, the addition of vanadium pentoxide was continued, accompanied by the pumped introduction of 805 kg of 105% phosphoric acid. Following the addition of the phosphoric acid, the reaction mixture was heated under reflux to about 100 to 108° C. and left under these conditions for 14 hours. The suspension was then drained off into a pressure suction filter, which had been rendered inert with nitrogen and heated beforehand; the line to the filter was flushed with 200 kg of isobutanol and the suspension was filtered at a temperature of approximately 100° C. under a pressure above the suction filter of up to 0.35 MPa abs. The end of filtration was reached when the liquid level in the filtrate vessel increased by less than 0.5% over the course of 20 minutes, and a filtrate volume of approximately 5.0 m³ had been reached, this corresponding arithmetically to a filtercake residual isobutanol content of approximately 35%. The filtercake was then blown dry by continuous introduction of nitrogen at 100° C., with stirring using a centrally arranged, height-adjustable stirrer, over the course of about one hour. After the product had been blown dry, the vessel was heated to a jacket temperature of 170° C. and evacuated to a pressure of 10 kPa abs (100 mbar abs). After a drying time of 110 min under reduced pressure, the reduced pressure was broken by lean air (nitrogen/oxygen mixture with an oxygen content of about 6% by volume) and the jacket temperature was raised to 200° C. Thereafter a volume flow of approximately 30 m³/h (stp) of lean air was passed through the filtercake from bottom to top until the residual isobutanol content reached was <0.1% by weight.

The organic carbon, inorganic carbon and total carbon contents of the dried catalyst precursor powder were 2.3%, <0.01%, and 2.3% by weight, respectively.

### Example 1b

### For Comparison, in Analogy to Example of WO 03/078058 A1

An 8 m³-steel/enamel stirred tank, rendered inert with nitrogen, externally heatable by way of pressurized water, and containing flow breakers was charged with 4602 kg of isobutanol. After the three-stage impeller stirrer had been started up, the isobutanol was heated to 90° C. under reflux. On reaching this temperature, the addition of 690 kg of vanadium pentoxide was commenced by way of the conveying screw. When, after about 20 minutes, about ⅔ of the desired amount of vanadium pentoxide had been added, the addition of vanadium pentoxide was continued, accompanied by the pumped introduction of 805 kg of 105% phosphoric acid. Following the addition of the phosphoric acid, the reaction mixture was heated under reflux to about 100 to 108° C. and left under these conditions for 14 hours. The suspension was then drained off into a pressure suction filter, which had been rendered inert with nitrogen and heated beforehand and at a temperature of approximately 100° C. and under a pressure above the suction filter of up to 0.35 MPa abs. The filtercake was blown dry by continuous introduction of nitrogen at 100° C., with stirring using a centrally arranged, height-adjustable stirrer, over the course of about one hour. After the product had been blown dry, the vessel was heated to about 155° C. and evacuated to a pressure of 15 kPa abs (150 mbar abs). Drying was carried out until the dried catalyst precursor had a residual isobutanol content of <2% by weight.

The dried powder was then treated for 2 hours under air in a rotary tube having a length of 6.5 m, an internal diameter of 0.9 m and internal helical coils. The speed of rotation of the rotary tube was 0.4 rpm. The powder was conveyed into the rotary tube at a rate of 60 kg/h. The air supply was 100 m³/h. The temperatures of the five heating zones of equal length, measured directly on the outside of the rotary tube, were 250° C., 300° C., 345° C., 345° C. and 345° C.

The catalyst precursor powder thus treated had organic carbon, inorganic carbon, and total carbon contents of 0.7%, 0.5% and 1.2% by weight, respectively.

B) Shaping and Preactivation of the Catalyst Precursor Powders 1a and 1b

To produce the catalysts the corresponding catalyst precursor powder was mixed with 20% by weight of malonic acid and 1% by weight of graphite (based in each case on the catalyst precursor powder) and the mixture was compacted. The mixture was shaped using a further 1% by weight of graphite (based on the original catalyst precursor powder) in a tableting machine, to form hollow cylinders having dimensions of 6.5 mm×4.2 mm×3.7 mm (external diameter x height x inner-hole diameter). To produce the rings, pressing forces of approximately 14 kN were set for the tableting procedure. The corresponding hollow cylinders were subsequently calcined under the following conditions:
A) Installation of the catalyst at room temperature
B) Heating of the catalyst in air at 1 K/min to 150° C.,
C) Holding of the temperature for 60 min,
D) Change of the gas composition to a mixture consisting of 5% O₂, 45% N₂ and 50% H₂O, and heating at 1 K/min to 220° C.,
E) Holding of the temperature for 30 min,
F) Heating of the catalyst at 2 K/min to 390° C.
G) Holding of the temperature: precursor powder from example 1a: 32 min, precursor powder from example 1b: 12 min
H) Change of the gas composition to a mixture consisting of 50% N₂ and 50% H₂O and heating at 2 K/min to 425° C.,
I) Holding of the temperature for 180 min
J) Change of the gas composition to N₂, and cooling to room temperature.

C) Catalytic Testing

Process for Preparing Maleic Anhydride from n-butane Using a Model-Tube Experimental Plant

The experimental plant was equipped with a feed unit and a reactor tube. The plant was operated in single pass mode, as described in EP-B 1 261 424. The hydrocarbon was added in liquid form under flow control via a pump. Air was added, under flow control, as the oxygenous gas. Triethyl phosphate (TEP) was added in liquid form, in solution in water, again under flow control. The oxygen concentration was set to the desired value through the addition of nitrogen under flow control.

The length of the reactor tube was 6.5 m, and its internal diameter was 22.3 mm. Within the reactor tube, a multiple thermocouple having 20 temperature measurement points was located within a protective tube having an external diameter of 6 mm. The reactor was heated by means of a heat transfer circuit having a length of 6.5 m. The heat transfer medium used was a salt melt.

The reaction gas mixture was passed through the reactor tube from top to bottom. The upper 0.2 m of the 6.5 m long reactor tube remained unfilled. The next zone was a 0.3 m long preheating zone, filled with steatite moldings as inert material. Downstream of the preheating zone was the catalyst bed, containing a total of 2180 ml of catalyst. Immediately after the shell and tube reactor unit, gaseous product was withdrawn and passed to the on-line gas chromatography analysis system. The main flow of the gaseous reactor discharge was expelled from the plant. The measurements were made after a minimum catalyst run time of 150 h. The results are set out in table 2.

### DEFINITIONS

TEP triethyl phosphate
pᵢₙ reactor entry pressure
GHSV total gas flow in liters per liter of catalyst per hour (gas hourly space velocity)
T_{reactor} [° C.] temperature in the reactor
Y_{MAn} [%] yield of maleic anhydride

TABLE 2
Pilot tube testing (2% by volume of n-butane, GHSV = 2000 h⁻¹, 3% by
volume H₂O, 2.25 ppm by volume TEP, pᵢₙ = 2.3 bar gauge)

| | Inventive (catalyst | Comparative (catalyst |
|---|---|---|
| | precursor prepared | precursor prepared |
| Catalyst | as per example 1 a) | as per example 1 b) |
| V-Ox | 4.19 | 4.13 |
| BET surface area [m²/g] | 30.3 | 27.6 |
| Conversion [%] | 85.1 | 85.5 |
| T_{reactor} [° C.] | 401 | 407 |
| Y_{MAn} [mol %] | 58.0 | 57.2 |
| Y_{MAn} [% by weight] | 97.8 | 96.5 |

It can be seen from table 2 that the inventive catalyst has a higher activity (lower salt bath temperature T_{reactor}) and yet a better selectivity (higher yield of maleic anhydride, Y_{MAn}) than the comparative catalyst.

1-10. (canceled)

11. A process for preparing a vanadium, phosphorus, and oxygen comprising catalyst precursor for preparing maleic anhydride by heterogeneously catalyzed gas-phase oxidation of a hydrocarbon having at least four carbon atoms, which comprises
(a) reacting vanadium pentoxide with 102% to 110% strength phosphoric acid in the presence of isobutanol and optionally of a primary or secondary, noncyclic or cyclic, unbranched or branched, saturated alcohol having 3 to 6 carbon atoms in a temperature range from 80 to 160° C.;
(b) isolating the precipitate formed;
(c) (i) drying the isolated precipitate down to a residual isobutanol content of less than 5% by weight;
(ii) then passing a gas which in addition to one or more inert gases comprises 0.1% to 9% by volume of oxygen through the dried precipitate, directly or after isolation, in the temperature range from 130° C. to 200° C.

12. The process according to claim 11, wherein the catalyst precursor obtained after step (c) has an organic carbon content in the range from 1 to 5% by weight.

13. The process according to claim 11, wherein the catalyst precursor obtained from step (c), following the addition of 3.0% by weight of graphite as internal standard and using CuKa radiation (π=1.54×10-10 m), gives a powder X-ray diffraction diagram which in the 2θ region features a ratio of the height of the peak of any hemihydrate phase present at 30.4° to the height of the peak due to the graphite at 26.6° of at least 2.0.

14. The process according to claim 11, wherein the alcohol component in step (a) comprises isobutanol in an amount of 90 to 100% by weight.

15. A process for preparing a shaped vanadium, phosphorus, and oxygen comprising catalyst precursor for preparing maleic anhydride by heterogeneously catalyzed gas-phase oxidation of a hydrocarbon having at least four carbon atoms, which comprises
(a) reacting vanadium pentoxide with 102% to 110% strength phosphoric acid in the presence of isobutanol and optionally of a primary or secondary, noncyclic or cyclic, unbranched or branched, saturated alcohol having 3 to 6 carbon atoms in a temperature range from 80 to 160° C.;
(b) isolating the precipitate formed;
(c) (i) drying the isolated precipitate down to a residual isobutanol content of less than 5% by weight;
(ii) then passing a gas which in addition to one or more inert gases comprises 0,1% to 9% by volume of oxygen through the dried precipitate, directly or after isolation, in the temperature range from 130° C. to 200° C.; and
(d) shaping the product obtained from step (c) into particles having an average diameter of at least 2 mm,

16. The process according to claim 15, wherein particles of substantially hollow cylindrical structure or multihole structure are shaped in step (d).

17. A catalyst precursor for preparing maleic anhydride by heterogeneously catalyzed gas-phase oxidation of a hydrocarbon having at least four carbon atoms, obtainable by the process defined in claim 11.

18. A process for preparing a vanadium, phosphorus, and oxygen comprising catalyst for preparing maleic anhydride by heterogeneously catalyzed gas-phase oxidation of a hydrocarbon having at least four carbon atoms by treating a vanadium, phosphorus, and oxygen comprising catalyst precursor in at least one atmosphere comprising oxygen (O₂), hydrogen oxide (H2O), and/or inert gas in a temperature range from 250 to 600° C., which comprises using the catalyst precursor according to claim 17.

19. A catalyst for preparing maleic anhydride by heterogeneously catalyzed gas-phase oxidation of a hydrocarbon having at least four carbon atoms, obtainable by the process as defined in claim 18.

20. A process for preparing maleic anhydride by heterogeneously catalyzed gas-phase oxidation of a hydrocarbon having at least four carbon atoms with oxygenous gases, which comprises using the catalyst as defined in claim 19.
